(19) United States
(12) Patent Application Publication
(10) Pub. No.: US 2005/0075621 A1
(43) Pub. Date: Apr. 7, 2005

(54) Method and apparatus for the ultrasonic cleaning of biofilm coated surfaces

(76) Inventor: Michael Rontal, Bloomfield Hills, MI (US)

Correspondence Address: GIFFORD, KRASS, GROH, SPRINKLE & CITKOWSKI, P.C, PO BOX 7021, TROY, MI, 48007-7021, US

(21) Appl. No.: 10/955,173

(22) Filed: Sep. 30, 2004

Related U.S. Application Data
(60) Provisional application No. 60/508,824, filed on Oct. 3, 2003.

(51) Int. Cl. A61M 31/00

(52) U.S. Cl. 604/500; 604/22

(57) ABSTRACT

To treat body surfaces, such as the sinuses, which are coated with biofilms, the surface is irrigated and suctioned with a fluid which may contain a biocide or other chemical agent for disrupting the biofilm while ultrasonic energy is applied to the fluid barrier formed over the biofilm. Action of the fluid enhanced by the ultrasonic energy tends to remove sections of biofilm which are suctioned out of the site. An electrical field may also be applied to the biofilm to enhance the disruptive action. Apparatus for practicing this method to treat chronic rhinosinusitis comprises an elongated tube adapted to be inserted into sinus cavities through the nose or mouth. The tube includes a first lumen which feeds an irrigating fluid containing biocides and/or biofilm-disruptive chemicals to the treatment site and a second lumen which suctions fluid from the site. An ultrasound horn extends through the tube and its distal end introduces ultrasonic energy into the fluid layer overlying the biofilm. In an alternative embodiment, the tube includes a pair of electrodes which establish an electric field across the biofilm, accelerating degradation.

### RELATED APPLICATION

This application claims priority of U.S. Provisional Patent Application Ser. No. 60/508,824 filed Oct. 3, 2003, which is incorporated herein by reference.

### FIELD OF THE INVENTION

This invention relates to methods and apparatus for the ultrasonic cleaning of bodily tissues coated with biofilm and more particularly, to such method and apparatus employing irrigation and suction to create a fluid layer over the biofilm and the application of ultrasonic energy to the biofilm through the fluid layer.

### BACKGROUND OF THE INVENTION

Bacteria may exist within a fluid media in a planktonic state or may form on a surface bounding the fluid medium in a conglomerate of microbial organisms termed a biofilm. In the biofilm, the bacteria live at a lower metabolic state than when in planktonic form and exude a hydrated matrix of exopolymers, typically polysaccharides, and other macromolecules. Bacteria in the biofilm form strong chemical bonds with surface carbohydrate moieties. The exopolymers encase the bacteria in a manner that leaves tunnels or channels through which the overlying fluid medium can circulate. In this way, the bacteria are protected from the dangers of the fluid medium, can receive nutrients, and rid themselves of waste. The protective film formed as part of a biofilm shields the bacteria from the action of antimicrobials and like-therapeutic agents at concentrations which would otherwise normally affect the bacteria.

The bacteria in this unique metabolic state affect other bacteria in the region to produce a coordinated lifestyle. This process is termed “quorum sensing.”

Biofilms may be formed on the surface of any living tissue, as well as foreign bodies, such as heart valves and the like, which are maintained in association with human tissues. When the biofilm is formed on living tissue, the biochemical products and toxic wastes it secretes may affect the tissue surface to produce an inflammatory state and areas of chronic infection, such as chronic ear disease, osteomyelitis, chronic tonsillitis, prostatitis, vaginitis, and calculi, as in the kidney. In many cases, chronic sinusitis appears to be an inflammatory disease of the lining mucosal, rather than the disease of bacteria-invading tissue. I have conducted electron microscopic studies that show biofilm exists on the mucosal surface. Collateral damage from the immune interaction between the biofilm products and the associated tissue would be the basis of the inflammatory mucositis seen in chronic rhinosinusitis.

The biofilm insulates the embedded bacteria from biocides contained in the proximal fluid layer so that normal concentrations of antibiotics or the like, which would kill the bacteria if they were in a planktonic state, have little or no effect on the bacteria of a biofilm. Antibiotic concentrations of 1000 to 2000 times higher than possible with systemic applied antibiotics would be required to destroy the bacteria of a biofilm.

Past efforts to disrupt the biofilm by breaking it up or killing the bacteria have included treatment with chemical compounds such as antibiotics, chemical agents directed at dissolving or breaking up the polysaccharide binders such as surfactants, enzymes, denaturing agents, and the like. In the dental field, the most effective treatment has been found to be scraping and debriding with mechanical instruments. Efforts have also been made to use ultrasonic energy to either increase the metabolic rate of the underlying bacteria so that they better absorb antibiotics and the like, or to mechanically disrupt the biofilm encasement by the mechanical bursting of micro-bubbles induced by ultrasonic energy sources. It has also been suggested that electric fields imposed across the biofilms or the fluid layers in contact with the biofilm will enhance break-up or electrophoretically drive biocides into the bacteria encased in the layers.

### SUMMARY OF THE INVENTION

The present invention is accordingly directed toward a method of removing biofilms in general, and particularly from living tissue, and more particularly from body cavities that are coated with biofilm, by flowing fluid containing various biofilm-active agents against the biofilm and suctioning the fluid from the area as ultrasonic energy is applied to the fluid. This fluid irrigation is introduced under pressure and withdrawn by a suctioning action to introduce the disruptive materials to the biofilm and the ultrasound produces shear forces which tend to tear off portions of the film and withdraw them from the treatment area.

This irrigation-suction action creates a fluid film over the biocide and ultrasonic energy is introduced into the film to mechanically drive the fluid into the film and produce micro-bubbles in the fluid which release energy upon bursting and mechanically disrupt the fluid. Alternatively, the ultrasonic energy may increase bacterial metabolism leading to susceptibility to deranging protein synthesis or cell division. In certain embodiments of the invention which will subsequently be described in detail, this irrigation/suction accompanied by ultrasonic energy introduced into the resulting film may be accompanied by electric fields imposed across the biofilm or the fluid interfacing the biofilm and/or mechanical scrubbing, to further enhance the breakup of the biofilm.

These actions to disrupt the biofilm are all designed in such a way as to neither destroy nor unduly stress the underlying tissue.

A preferred embodiment of the apparatus for practicing the present invention, which will subsequently be described in detail, comprises an elongated tube or barrel, adapted to be introduced to the human body through the nasal passages or otherwise, so that its distal end is in proximity to a biofilm-lined sinus to be treated. The tube may be rigid or flexible, straight or bent, and includes a first lumen for introducing pressurized bio-treatment fluid at the proximal end so that it passes through the tube and exits at the distal end. The discharge may be through a nozzle to produce a high-velocity spray. A second lumen is connected to a vacuum source at the proximal end so as to create a suction at the distal end to remove excess fluid along with debris, including fragments from the biofilm and secretions from the sinuses. Both the irrigation of the bio-affecting fluid and its suctioned removal may be continuous or intermittent, controlled by valves. This allows the introduction of fluid pressure waves by the alternate introduction of pressured fluid and its suctioned removal.

The distal section of the tube may be manually deformable to allow the surgeon to conform the tube to particular applications. This distal section may be removable from the main section of the apparatus to allow replacement with a sanitary, unbent section.

In an alternative embodiment of the invention the ultrasonic energy is introduced to the distal end of the application tube by introducing the ultrasound into the proximal end of the irrigating lumen so that the fluid column in the tube carries the ultrasonic forces to the treatment area, eliminating the need for an ultrasound horn formed along the length of the apparatus.

In an alternative embodiment of the invention, the biofilm affected tissue may be encased in a chamber having open resilient edges which bear against the tissue at its boundaries; the bio-affecting fluid is then introduced and removed from the chamber and ultrasonic forces are imposed on the fluid contained within the chamber, and bearing against the biofilm, either by a ultrasonic horn projecting into the fluid-filled cavity, or by the application of ultrasonic forces to the wall of the chamber.

The biofilm encasing chamber may either be formed at the end of an elongated tube containing the fluid lumens and the ultrasonic horn, or as a separate device which may be applied to external body parts, such as skin burns.

Other objectives, advantages and applications of the present invention will be made apparent by the following detailed description of several embodiments of the invention. The descriptions make reference to the accompanying drawings in which:

### BRIEF DESCRIPTION OF THE DRAWINGS

FIG. 1 is a perspective view of a handheld instrument, formed in accordance with the present invention, for practice of the inventive method;

FIG. 2 is a cross-sectional view of the tube of the handheld tool of FIG. 1, taken along line 2-2 of FIG. 1;

FIG. 3 is a cross-sectional view of the device of FIG. 1 inserted into a living body cavity, with sections broken away to show the construction of the tube;

FIG. 4 is a cross-sectional view of an alternative embodiment of the apparatus of the present invention inserted into a living body cavity, and partially broken away to exhibit the electrodes used to impose an electrical field across the biofilm;

FIG. 5 is a cross-sectional view of another alternative embodiment of the apparatus of the present invention which includes a biofilm abrading device for imparting mechanical energy to the biofilm, supported at the distal end of the tube;

FIG. 6 is a view, partly in section, of an alternative form of the apparatus of the present invention including a cavity adapted to surround the treatment area; and

FIG. 7 is another alternative embodiment of the apparatus of the present invention including a cavity adapted to surround the treatment area and having inlet and outlet ports for the bio-reducing agent and means for introducing ultrasonic energy through the wall of the cavity.

### DETAILED DESCRIPTION OF THE INVENTION

The method of the present invention broadly involves treatment of a body tissue having a biofilm coating on its surface by irrigating the surface with a flow of fluid and suctioning the excess fluid off while imparting energy to the biofilm directly or through the fluid to reduce or change the biofilm. The irrigating fluid preferably contains a bio-reducing agent which will reduce or disrupt the biofilm by destroying its integrity or damaging the constituent bacterial cells. These agents may include surfactants, proteases, enzymes, denaturing agents, and the like. They may include biocides such as antibiotics and antifungal agents.

The chemical agents which may disrupt and destroy the biofilm include guaifenesin, dornase alfa and N-acetylcysteine. These materials are particularly advantageously used in a preferred embodiment of the invention in which the biofilm and mucous coats the sinuses. Guaifenesin is a mucolytic and is often used for the treatment of sinusitis and rhinitis. Dornase alfa (ymogen) is used to treat the thick mucous of cystic fibrosis and N-acetlcysteine is used for excess mucous in chronic bronchitis. They are know to break up mucous which is involved in biofilm infections and may act on the biofilm itself. Thus, the use of these chemicals in the method of the present invention performs a synergistic role in simultaneously treating the underlying mucosal tissues and reducing the integrity of the overlying biofilm.

Any other bio-reducing or biocide drugs or combinations thereof may be used in a particular application.

The ultrasonic energy imparted into the fluid film covering the biofilm, in the practice of the present invention, may be of a sinusoidal or pulsed character. The ultrasonic signal is generated by a unit that is external of the body. The generator may be of a fixed frequency or it may scan a range of frequencies continually to ensure optimum coupling of energy through the fluid layer into the biofilm. The exact manner in which ultrasonic forces enhance destruction of biofilm may involve the physical agitation of the minute bubbles produced by the ultrasound in the overlying fluid. Bursting of these bubbles produces forces that may cause tears in the biofilm. Alternatively, the ultrasonic energy may increase the metabolism of the bacteria in the biofilm, increasing its susceptibility to the biocides and bio-reducing agents in the irrigating fluid. The energy of the microwave must be limited to avoid damage to the underlying tissues, and values as high as 250 watts per square centimeter are apparently safe. This device is not designed to destroy mucosal tissue. Relatively low frequencies have been found more effective than higher frequencies in ultrasonic treatment of biofilm and 10 kHz-100 kHz may be a reasonable range of application.

In those embodiments of the invention in which an electric field is applied across either the microfilm or the fluid layer overlying the microfilm, either AC or DC may be applied. The DC may be pulsed so that rapid changes in the field gradient induce tearing forces in the biofilm.

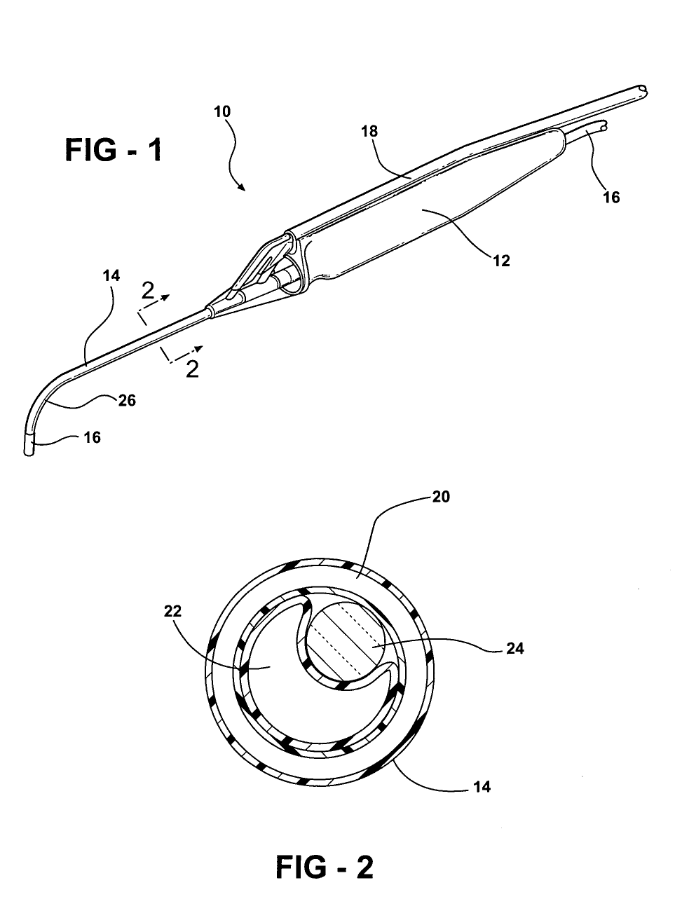

A preferred embodiment of an instrument for use in practice of the present invention is illustrated in FIG. 1. The instrument, generally indicated at 10, has a handle section 12 for manual support and manipulation of the device and an elongated application tube or barrel 14 extending from the handle and terminating in a distal end 16. The tube 14 may be rigid and may be straight or formed with a bend along its length. Alternatively, it may be made of a manually deformable material and may be bent as needed for application into a body cavity. The distal end of the tube 14 may be removable from the handle 12 for replacement.

A pair of conduits 18 extend along the handle and connect at their proximal end to a source of the bio-affecting irrigating fluid and to a sink for the suctioned fluid. (Not Shown) The fluid is pumped outwardly from the proximal end from a source in one conduit and is then carried by the other conduit back from the irrigated source to the proximal end.

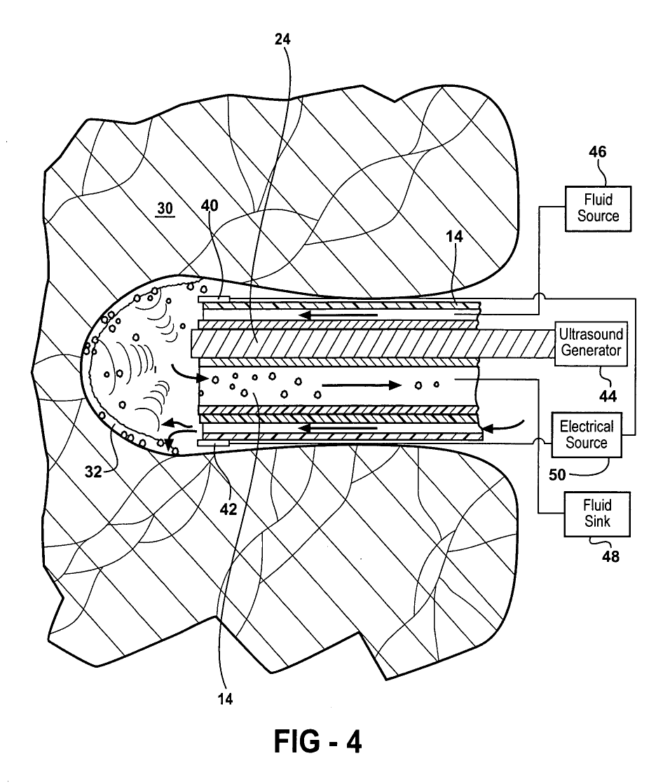

The pump which feeds the irrigating fluid to the instrument 10 and the suction device that retrieves it from the irrigated area may feed from the same sump with an appropriate filter in the return line to remove solid matter contained in the fluid. Alternatively, the fluid may not be reused and the irrigated fluid may be discarded. The two conduits 18 feed to lumens in the tube section 14. As is best seen in the cross section of FIG. 2, the irrigating fluid may pass through a lumen 20 which is concentric about the tube 14 along its length and return through a larger lumen 22. An ultrasound horn 24 carries energy from a generator 50 (FIG. 4) at the proximal end to the distal end.

When used for the treatment of rhinitis, the tube 14 is applied through the nasal cavity so that its proximal end is adjacent to the sinus area coated with biofilm to be treated. Irrigating fluid is then supplied, through lumen 20 and withdrawn through lumen 22 at a suitable rate to maintain a fluid layer over the biofilm area. Ultrasonic energy is then applied through horn 24 to the fluid layer so that forces are imposed on the biofilm.

The irrigation produces shear forces which tend to tear the protruding sections of the biofilm away and the mechanical agitation produced by the ultrasonic energy enhances this tearing action. The bio-affecting agents in the circulating fluid also act on the biofilm so as to reduce or remove it.

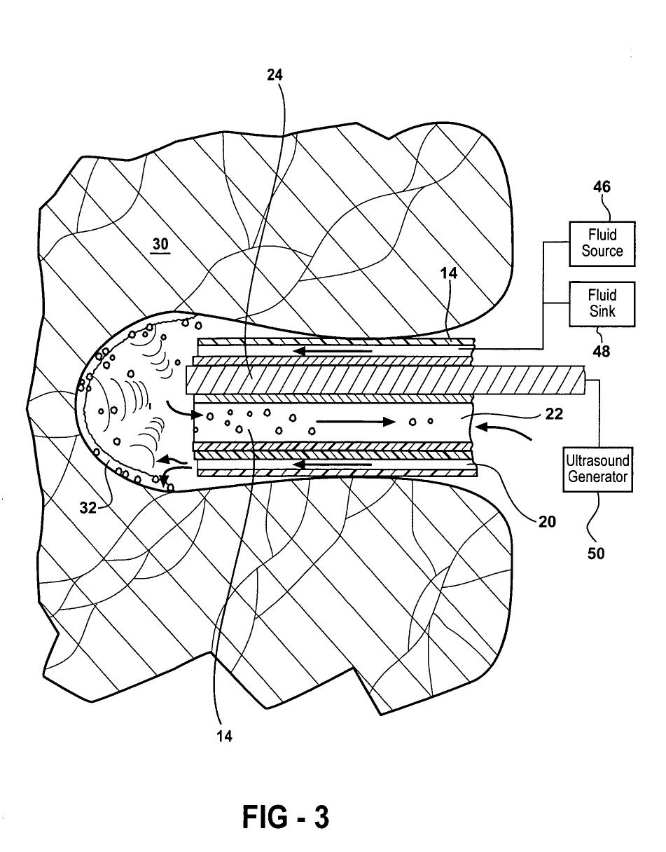

FIG. 3 illustrates the application of the method to a body cavity 30 such as the sinuses. A biofilm coating 32 extends over an infected area, releasing materials which inflame the underlying tissue. Irrigating fluid containing biocides and/or bio-reducing agents are introduced through the lumen 20 from a fluid source 46 and withdrawn from the larger area lumen 22 to a fluid sink 48. Ultrasonic energy is introduced into the fluid film which results from the irrigation via the ultrasonic horn 24 from a generator 50. The biofilm is acted on by the physical shearing forces imposed by the irrigation and suction; by the mechanical forces generated in the overlying fluid film from the ultrasound; and chemical action takes place as a result of the agents contained within the irrigating fluid. These factors reduce or completely eliminate the biofilm so as to free the inflamed area for application of antibiotics and the like which may be contained in the irrigating fluid or may be introduced separately following treatment with the irrigating fluid and ultrasound. The ultrasonic generator 50 provides the energy to the horn either at a set frequency or a scanned frequency or in pulses.

FIG. 4 illustrates an alternative embodiment of apparatus capable of imposing an electric field across the biofilm encoating the infected area and/or the fluid layer overlying the biofilm. The structure of the application tube is identical to the device in FIG. 1 with the exception that a pair of electrodes 40 and 42 extend down diametrically opposed sides of the tube from the proximal end to the distal end. At the proximal end they are connected to an electrical source 44 which generates a potential difference across the electrodes 40 and 42. The applied voltage may be either direct current, either constant or pulsed, or alternating current of a fixed or scanned frequency. The application device also connects to a fluid source 46, a fluid sink 48, and an ultrasound generator 50.

The electric field imposes phoretic forces on the biofilm and may drive the irrigating fluid into the biofilm to enhance disruptive action.

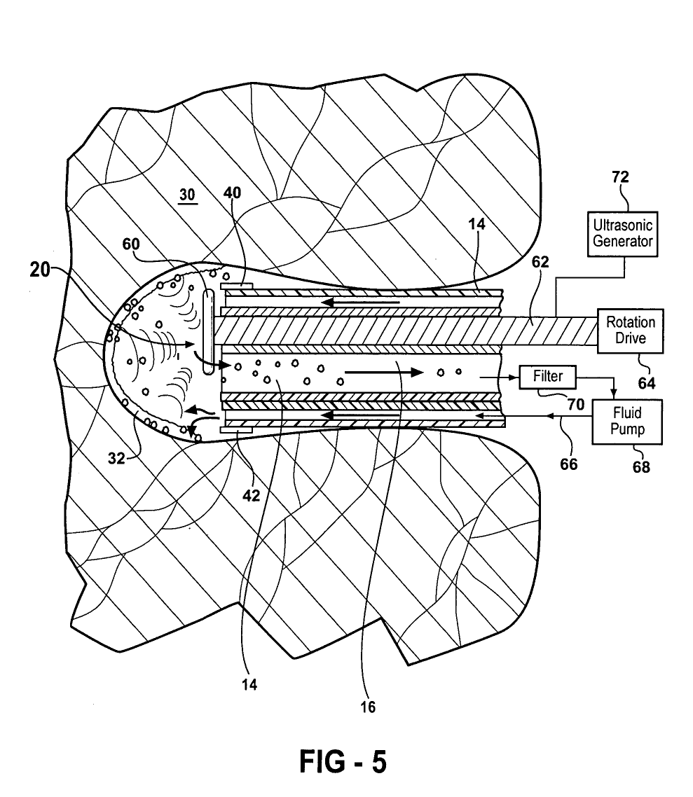

An embodiment of the invention illustrated in FIG. 5 applies mechanical forces to the biofilm through a brush or abrading device 60. The device is either rotated or oscillated through a flexible shaft 62 which extends through the center of the rod 14. At the proximal end it is driven by a drive member 64. Irrigating fluid is provided through a line 66 from a sump to the lumen 20 of the tube 14 and is returned through the lumen 22 to the sump 68 through a filter 70. Ultrasonic forces may also be applied through an ultrasonic horn driven by the generator 72.

Alternatively, the ultrasonic forces could be applied to the proximal end of the fluid column formed in the lumen 20 so that the ultrasonic energy is carried to the distal end 16 by that column, eliminating the need for an ultrasonic horn. The transmission of ultrasonic forces throughout a fluid column is described in ULTRASONICS, VOL26, No. 1, 1988 at pages 27-30. The electric field applying electrodes 40 and 42 of the embodiment of FIG. 4 could also be combined with this unit.

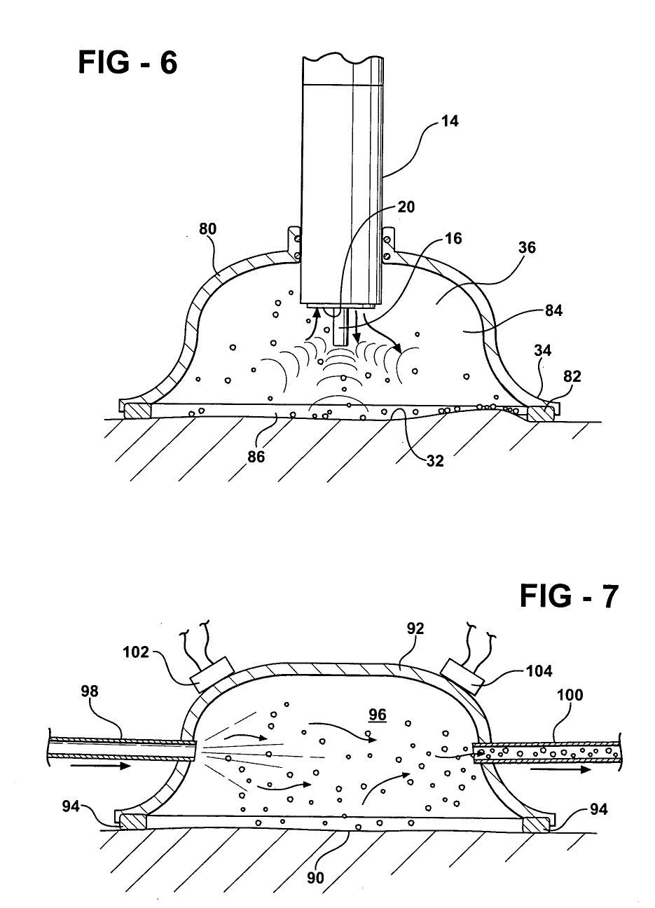

The method of the present invention may also be employed on living body tissues that are easily accessible, such as the outer body covered by skin or the mucous membranes of the oral areas. FIG. 6 illustrates an alternative embodiment of the apparatus of the present invention which can be used to treat biofilms formed on these accessible areas. A typical application is to treat a burned portion of the skin over which a biofilm has formed. The apparatus illustrated in FIG. 6 is substantially identical to the embodiment of FIG. 1 except for the provision of a semispherical cavity 80 which is attached to the rod 14 adjacent its distal end 20. The cavity has a central hole through which the distal end of the rod 14 passes so that the open end of the cavity extends beyond the distal end 20. A resilient gasket 82 is formed about the open edge of the cavity 80. By proper manipulation of the tube 14 the gasket may be pressed against an area of the skin to be treated to produce a closed containment volume 84.

The irrigating flow of fluid containing a biocide or other bio-affecting agent from the rod end 14 fills the volume 84 with fluid. As additional fluid is introduced the surplus is sucked off through the second lumen of the rod 14. Ultrasonic energy is then introduced into the fluid through the horn end 16, causing forces to be imposed on the treatment area 86 bounding the volume 84.

1. The method of reducing a biofilm, comprising:
irrigating the biofilm with a fluid containing a biofilm-reducing agent;
suctioning the biofilm-reducing agent from the treatment area, so as to maintain a fluid layer of the fluid in contact with the biofilm area to be treated; and
inducing ultrasonic energy into the fluid layer.

2. The method of claim 1 wherein the biofilm is disposed on the surface of human tissue.

3. The method of claim 2 where the human tissue comprises a body cavity.

4. The method of claim 3 wherein the body cavity constitutes the sinus cavity.

5. The method of claim 4 further comprising introducing and removing the fluid and introducing the ultrasonic energy into the fluid layer through an elongated tube having its distal end disposed within the body cavity and its proximal end exterior of the body.

6. The method of claim 1, further comprising: establishing an electric field across the biofilm directly or through the fluid layer.

7. The method of claim 4 wherein the biofilm-reducing agent is chosen from the group consisting of guaifenesin, dornase alfa or N acetylcysteine or a derivative thereof to simultaneously attack the biofilm matrix and reduce the mucous layer on the sinus lining.

8. The method of reducing biofilm resident on human tissue comprising:
surrounding a section of the tissue coated with biofilm by a cavity having an open edge with a resilient gasket formed thereon;
introducing fluid containing a biofilm-reducing agent into the cavity;
suctioning excess fluid and biofilm residue from the cavity; and
applying ultrasonic energy into the fluid contained within the cavity to produce mechanical forces on the biofilm.

9. The method of claim 8 further comprising establishing an electric field across the biofilm or the fluid layering contact with the biofilm to further enhance biofilm-reductive action.

10. The method of claim 9 further comprising mechanically agitating the biofilm surface in contact with the fluid within the cavity by a mechanical instrument introduced into the cavity.

11. Apparatus for treating biofilm covered tissue forming part of a cavity within a living body comprising:
an elongated tube adapted to be inserted into the body so that its distal end is disposed within the cavity and its proximal end is exterior of the body, the tube containing a first lumen for introducing fluid into the cavity and a second lumen for suctioning fluid from the cavity and an ultrasonic horn;
a first port for introducing fluid containing biofilm treatment reducing agents into the proximal end of the first lumen;
a second port at the proximal end of the tube for applying a suction force to the second lumen to suction fluid from the cavity; and
an ultrasonic generator connected to the proximal end of the ultrasonic horn so as to introduce ultrasonic energy from the distal end of the tube into fluid contained within the cavity.

12. The apparatus of claim 11 further including a pair of electrodes extending along the tube between the distal and the proximate end, and an electric generator for applying an electric potential to the proximal ends of the electrodes so as to create a field across the biofilm or the fluid in contact with the biofilm within the cavity.

13. The apparatus of claim 12 wherein the tube is manually deformable.

14. The apparatus of claim 11 further including:
a source of mechanical motion connected to the proximal end of the tube;
a biofilm abrading device supported on the distal end of the tube; and
an elongated member extending through the tube connecting said source of mechanical motion to said biofilm abrading device to impart motion to the abrading device relative to the tube.

15. The apparatus of claim 14 in which the mechanical motion is rotational.

16. The apparatus of claim 14 in which the mechanical motion is vibratory.

17. The apparatus of claim 11 in which the ultrasonic generator introduces energy into the proximal end of the first lumen.

18. The apparatus of claim 11 in which the distal end of the elongated tube is manually deformable.

19. The apparatus of claim 111 in which the distal end of the elongated tube is removable from the proximal end for replacement purposes.
